(19) United States
(12) Patent Application Publication
(10) Pub. No.: US 2023/0135403 A1
(43) Pub. Date: May 4, 2023

(54) SURGICAL DEVICE SYSTEM

(71) Applicant: ARTHREX, INC., Naples, FL (US)

(72) Inventors: Richard John Taft, Naples, FL (US); Jeremiah Daniel Caldwell, Naples, FL (US)

(21) Appl. No.: 17/875,231

(22) Filed: Jul. 27, 2022

Related U.S. Application Data
(60) Provisional application No. 63/273,462, filed on Oct. 29, 2021.

(51) Int. Cl. A61B 17/32

(52) U.S. Cl. CPC A61B 17/32002; A61B 2217/005

(57) ABSTRACT

A surgical device having a body with a proximal end and a distal end; a hollow cannula extending from the distal end of the body, the cannula having a proximal end and a distal end; an operative head positioned at the distal end of the cannula; and a suction connector in fluid communication with the hollow cannula, the suction connector being configured for connection to a suction source; wherein the hollow cannula has at least one of the group consisting of diverters and rifling on at least a portion of an inside surface.

### CROSS REFERENCE TO RELATED APPLICATION

This application claims priority of U.S. Provisional Patent Application No. 63/273,462, filed on Oct. 29, 2021, entitled SURGICAL DEVICE SYSTEM, the entire contents of which is hereby incorporated herein by reference.

### BACKGROUND

The present disclosure relates to devices used in surgery and, more particularly, to flow paths through surgical devices.

Certain surgical devices, such as shavers, typically employ suction to remove fluid, tissue and other debris from a surgical site. Suction may also be employed to draw tissue into a surgical device such for cutting. The fluid, tissue and debris sucked into the surgical device travels through a channel and out of the surgical device for disposal. Sometimes the tissue and debris adhere to the side of the channel and, if enough tissue and debris builds up in the channel, then proper functioning of the device may be compromised.

Therefore, there exists a need for a system and method of improving fluid, tissue and debris flow through surgical devices that remedies the shortcomings of the prior art.

### SUMMARY

The present disclosure is directed to a surgical device with a divertors or rifling along at least a portion of a suction pathway to improve fluid, tissue and debris flow.

In an implementation, a surgical device has a body with a proximal end and a distal end. A hollow cannula extends from the distal end of the body, the cannula having a proximal end and a distal end. An operative head may be positioned at the distal end of the cannula. A suction connector is in fluid communication with the hollow cannula, the suction connector being configured for connection to a suction source. The hollow cannula has at least one of the group consisting of diverters and rifling on at least a portion of an inside surface.

The operative head may have a radio frequency ablator. The operative head may have diverters on at least a portion of an inside surface. The operative head may have rifling on at least a portion of an inside surface. In an implementation, the body has a suction bore extending from the suction connector to a position near the hollow cannula, the suction bore being in fluid communication with the suction connector and the hollow cannula; and wherein the suction bore further comprises diverters along at least a portion of an inside surface. In an implementation, the body has a suction bore extending from the suction connector to a position near the hollow cannula, the suction bore being in fluid communication with the suction connector and the hollow cannula; and wherein the suction bore further comprises rifling along at least a portion of an inside surface.

In an implementation, the hollow cannula may have diverters on at least a portion of an inside surface and the diverters may have a round profile. The diverters may be straight, curved, or in a helical pattern. The diverters may also be in a double helix pattern. In an implementation, the hollow cannula has rifling on at least a portion of an inside surface. The rifling may be in a curved or a helical pattern. The rifling may also be in a double helix pattern.

In an implementation, a surgical device has a body with a proximal end and a distal end. A suction connector is coupled to the body, the suction connector being configured for connection to a suction source. A hollow cannula extends from the distal end of the body, the cannula having a proximal end and a distal end. An inner shaft is rotatably positioned within the hollow cannula, the inner shaft having a proximal end, a distal end, and a channel. An inner drive hub is coupled to the proximal end of the inner shaft, the inner drive hub being rotatably coupled to the body and in fluid communication with the suction connector. The channel has at least one of the group consisting of diverters and rifling on at least a portion of an inside surface.

In an implementation, a suction bore extends from the suction connector to a position near the inner hub, the suction bore being in fluid communication with the suction connector and the channel of the inner shaft. The suction bore may have diverters along at least a portion of an inside surface. The suction bore may also have rifling along at least a portion of an inside surface.

In an implementation, the channel may have diverters on at least a portion of an inside surface and the diverters may have a round profile. The diverters may be in a helical pattern. The diverters may be in a double helix pattern. In an implementation, the channel may have rifling on at least a portion of an inside surface and the rifling may be in a helical pattern. The rifling may also be in a double helix pattern.

In an implementation, a cutting head may be positioned at the distal end of the hollow cannula; and a blade may be positioned at the distal end of the inner shaft. As the inner shaft is rotated relative to the cannula, the blade cooperates with the cutting head to cut tissue in contact with the cutting head. The surgical device may be selected from the group consisting of: shavers, drills, burrs and rasps.

These and other features are described below.

### BRIEF DESCRIPTION OF THE DRAWINGS

The features, aspects and advantages of the present disclosure will become better understood with regard to the following description, appended claims and accompanying figures wherein:

FIG. 1 is a schematic side elevation view of a surgical device according to an implementation;

FIG. 2 is a side elevation cut-away view of a portion of a surgical device configured as an ablation device according to an implementation;

FIG. 3 is a perspective cut-away view of a portion of a surgical device configured as an ablation device according to an implementation;

FIG. 4 is a cross sectional view of a channel of a surgical device configured as an ablation device according to an implementation;

FIG. 5 is a perspective cut-away view of a portion of a surgical device configured as an ablation device according to another implementation;

FIG. 6 is a cross sectional view of a channel of the surgical device of FIG. 5;

FIG. 7 is a perspective cut-away view of a portion of a surgical device configured as an ablation device according to another implementation;

FIG. 8 is a cross sectional view of a channel of the surgical device of FIG. 7;

FIG. 9 is a perspective cut-away view of a portion of a surgical device configured as an ablation device according to another implementation;

FIG. 10 is a schematic representation of a channel having a double helix pattern of divertors according to an implementation;

FIG. 11 is a side elevation cut-away view of a surgical device configured as a shaver according to an implementation; and

FIG. 12 is a side elevation cut-away view of a surgical device configured as a shaver according to an implementation.

### DETAILED DESCRIPTION

In the following description of the preferred implementations, reference is made to the accompanying drawings which show by way of illustration specific implementations in which the device may be practiced. Wherever possible, the same reference numbers will be used throughout the drawings to refer to the same or like parts. It is to be understood that other implementations may be utilized and structural and functional changes may be made without departing from the scope of this disclosure.

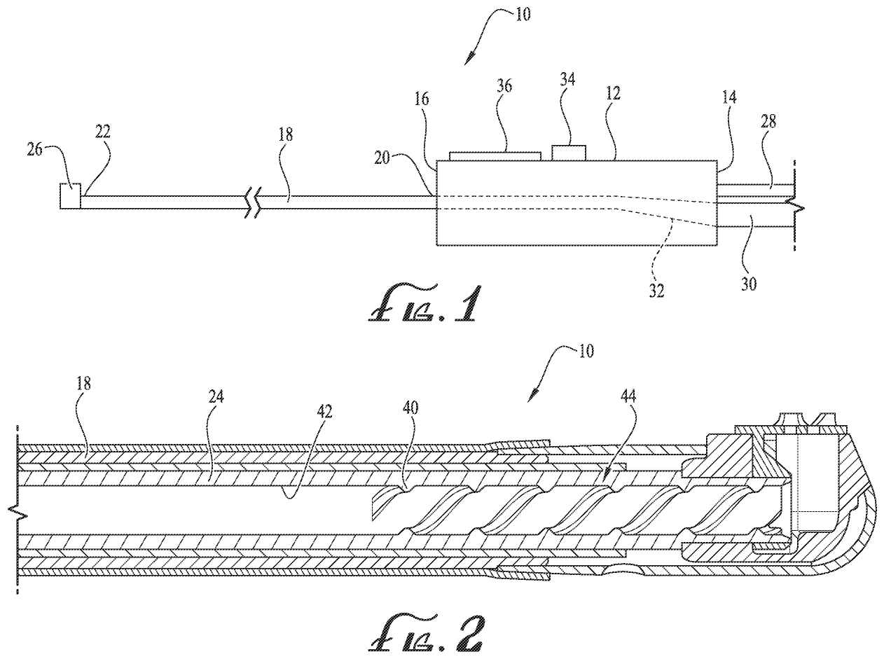

With reference to FIGS. 1 to 10, a surgical device 10 according to an implementation has a body 12. The body 12 has a proximal end 14 and a distal end 16. A cannula 18 may be coupled to the distal end 16 of the body 12. The cannula 18 has a proximal end 20 and a distal end 22. The cannula is hollow and has a channel 24 passing therethrough from the proximal end 20 to the distal end 22. An operative head 26 may be coupled to the distal end 22 of the cannula 18. The cannula 18 length may be varied depending on the desired application. In an implementation, the cannula 18 has a length of between about 50 mm and about 150 mm.

In an implementation, a power supply cable 28 may be coupled to the proximal end 14 of the body 12. In an additional implementation, the surgical device 10 is battery powered and there may be no need for a power supply cable. A suction connector 30 may be located on the proximal end 14 of the body 12. The suction connector 30 is in fluid communication with a suction source (not shown). The suction connector 30 is in fluid communication with a suction bore 32. The suction bore 32 extends from the suction connector to an area near the proximal end 20 of the cannula 18. The suction bore 32 is in fluid communication with the channel 24. The suction source is therefore in fluid communication with the suction connector 30, the suction bore 32 and the channel 24. Suction may be used to extract material from a surgical field through the operative head 26, through the channel 24 of the hollow cannula 18, through the suction bore 32 and out through the suction connector 30.

The operative head 26 may be configured as a cutting device such as, for example and without limitation, a mechanical cutter or a radio frequency ablation device. Additionally, the operative head 26 may be configured as a shaver. Additionally, the surgical device may be a simple suction device and the operative head may contain an opening for material to pass through. The body 12 may have an actuator 36 for triggering an action by the operative head 26. Additionally, the body 12 may have a display 38 for displaying at least one condition of the device 10.

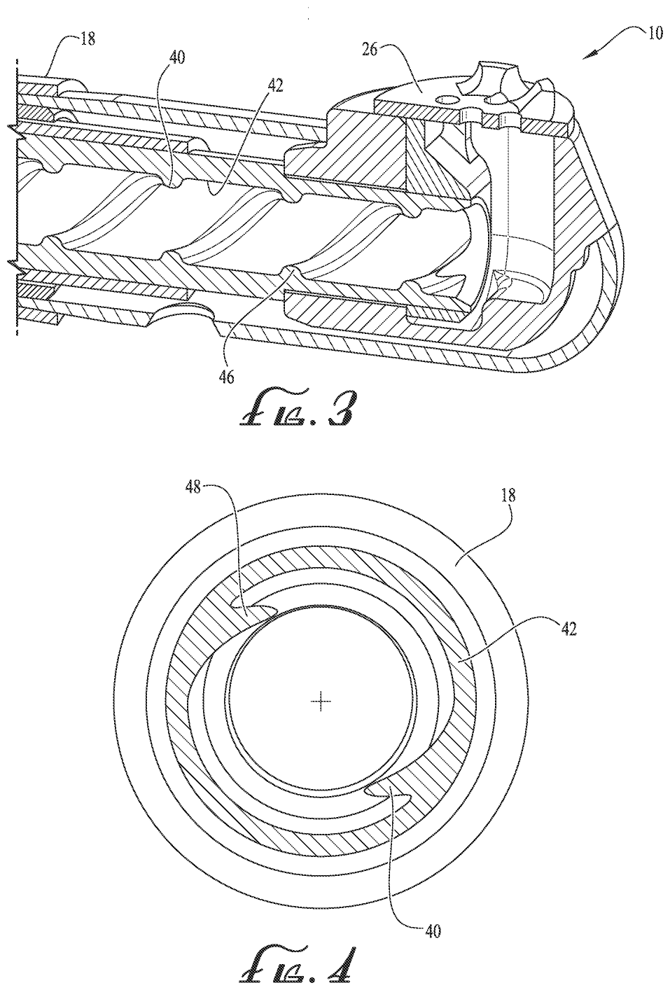

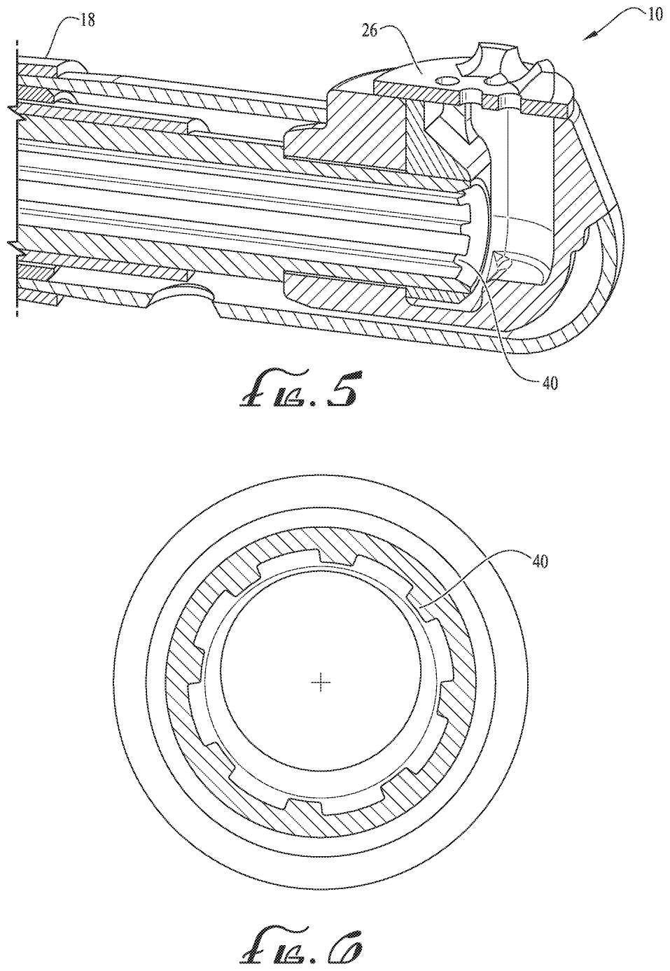

In an implementation, divertors 40 are placed on an inner surface where fluid, tissue or debris are evacuated through the surgical device 10. As used herein, the term “divertors” refers to protrusions extending from a surface 42. As shown in, for example, FIGS. 2 and 3, the divertors 40 may be placed in a spiral (helical) pattern 44. The divertors 40 may have a rounded profile 46. In other implementations, the divertors may have a bladed profile 48, such as shown in FIG. 4.

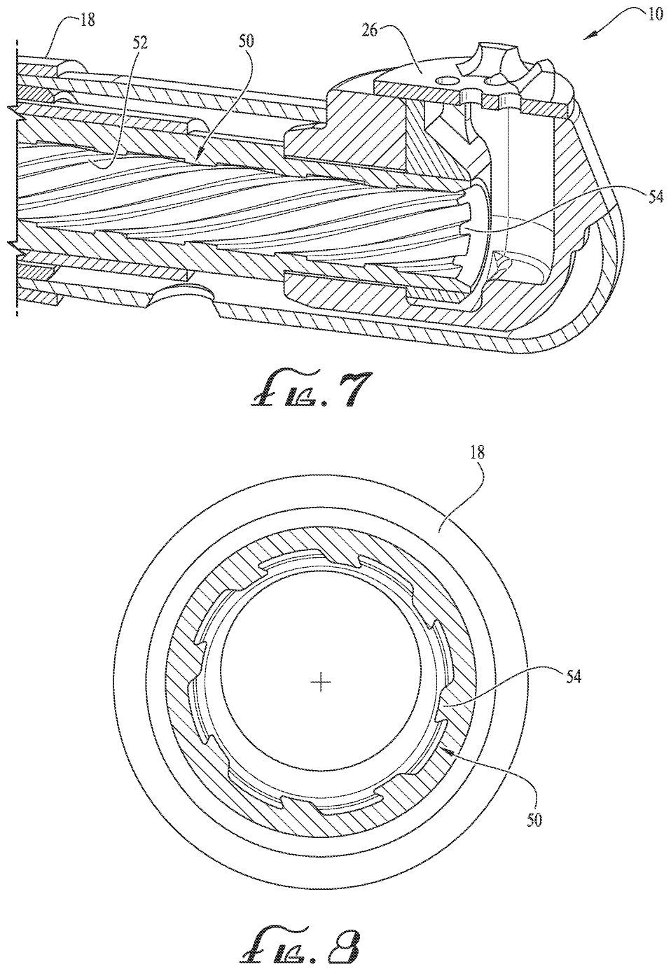

As shown in FIGS. 5 and 6, the divertors 40 may extend parallel to a longitudinal axis of the cannula 18 to form a corrugated tube. Tissue may flow on the divertors 40 while fluid may be travel into grooves between the divertors. The geometry of the divertors 40 may be altered depending on, for example and without limitation, the likely fluids, tissue and debris expected to pass through the device as well as the amount of suction expected to be used.

In an implementation, the divertors 40 are placed inside the channel 24, such as and without limitation in an area proximal to the distal end 22 of the cannula 18. As shown in FIG. 9, the divertors 40 may also be places on an inner surface of the operative head 26. The divertors 40 may also be placed in the suction bore 32, such as, and without limitation, proximal to the suction connector 30.

As fluid, tissue and debris contact the divertors 40, rotation may be induced. The rotation may help to further break up tissue and debris to prevent clumping and blockages. The divertors 40 direct fluid, tissue and debris within a fluid path to prevent fluid, tissue or debris from adhering to the surface 42 of the fluid path. The size and configuration of the divertors 40 may change along the length of the fluid path. The divertors 40 may be machined into the fluid path. Additionally, the divertors 40 may be molded into the fluid path, such as when the fluid path is formed of metal, plastic or ceramic. Additionally, the divertors 40 may be on an insert that is placed in the fluid path.

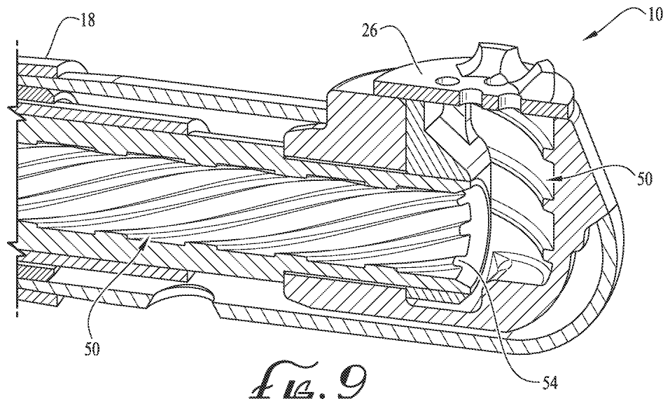

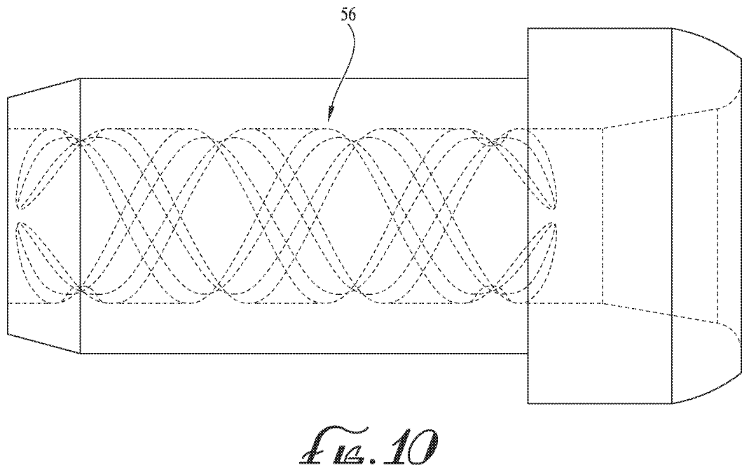

In an implementation, rifling 50 is placed on an inner surface where fluid, tissue or debris are evacuated through the surgical device 10. As used herein, the term “rifling” refers to grooves in the surface 42. In an implementation, as shown in for example in FIGS. 7 and 8, the rifling 50 is in a spiral (helical) pattern 52. In an implementation, the rifling 50 has a substantially rectangular profile. In other implementations, the rifling 50 may have a rounded profile. In an implementation, as shown in FIGS. 7 and 8, the rifling 50 has a polygonal profile 54.

In an implementation, the rifling 50 is placed inside the channel 24, such as and without limitation in an area proximal to the distal end 22 of the cannula 18. As shown in FIG. 9, the rifling 50 may also be placed on an inner surface of the operative head 26. The rifling 50 may also be placed in the suction bore 32, such as, and without limitation, proximal to connection of the suction source 30. The rifling 50 is used to exert torque and impart a spin to fluid, tissue or debris passing through the surgical device to prevent fluid, tissue or debris from adhering to the inner surface of the fluid path fluid for easier removal.

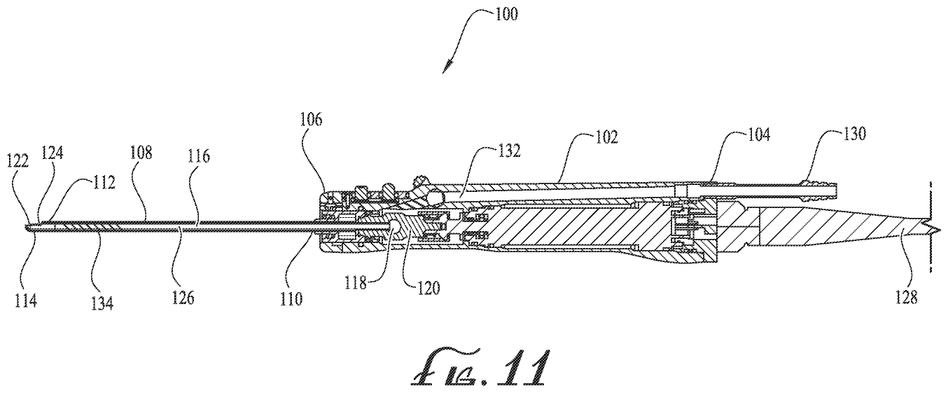

The rifling 50 may be machined into the fluid path. Additionally, the rifling 50 may be molded into the fluid path, such as when the fluid path is formed of metal, plastic or ceramic. Additionally, the rifling 50 may be on an insert that is placed in the fluid path. In an implementation, as shown in FIG. 10, the divertors 40 or the rifling 50 may be placed in a double helix pattern 56 to direct fluid/tissue and or dislodge and keep tissue from sticking to main inner lumen wall.

In an implementation, the surgical device 10 has both divertors 40 and rifling 50. Additionally, the divertors 40 or rifling 50 may be coated, such as with hydrophobic coatings. The coatings may be made using physical vapor deposition (PVD).

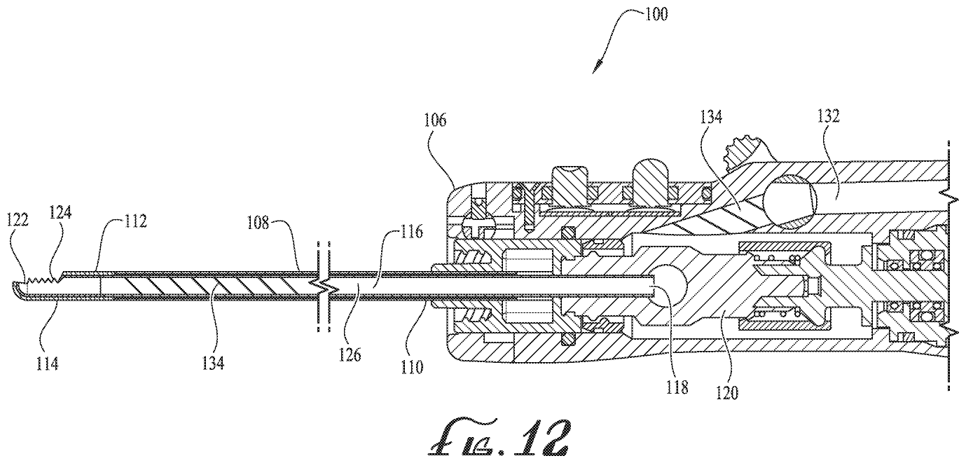

A surgical device 100 according to an additional implementation, namely a surgical shaver, is shown in FIGS. 11 and 12. As shown in FIGS. 11 and 12 the shaver 100 has a body 102, with a proximal end 104 and a distal end 106. A cannula 108 is coupled to the distal end 106 of the body 102. The cannula 108 has a proximal end 110 and a distal end 112. The cannula 108 is hollow. A cutting head 114 is coupled to the distal end 112 of the cannula 108. An inner shaft 116 is rotationally positioned within the cannula 108. The inner shaft 116 has a proximal end 118 coupled to an inner hub 120 and a distal end 122. The inner hub 120 is rotatably mounted within the body 102. The distal end 122 has a blade 124. As the inner shaft 116 is rotated relative to the cannula the blade 124 cooperates with the cutting head 114 to cut tissue in contact with the cutting head. The inner shaft 116 is hollow and contains a channel 126. Although a shaver with a cutting head 114 and blade 124 have been illustrated, the surgical device 100 may have other components on the distal ends of the cannula 108 and the inner shaft 116, and may be configured for example, and without limitation, as drills, burrs, and rasps.

A power supply cable 128 may be coupled to the proximal end 104 of the body 102. A suction connector 130 may be located on the proximal end 104 of the body 102. The suction connector 130 is in fluid communication with a suction source (not shown). The suction connector 130 is in fluid communication with a suction bore 132. The suction bore 132 extends from the suction connector to an area near the inner hub 120. The suction bore 132 is in fluid communication with the inner hub 120 and the proximal end 118 of the inner shaft 116. The suction source is therefore in fluid communication with the suction connector 130, the suction bore 132 and the channel 126. Suction is used to extract material from a surgical field through the cutting head 114, through the channel 126, through the suction bore 132 and out through the suction connector 130.

In an implementation, divertors 134 are placed inside the channel 126, such as and without limitation in an area proximal to the distal end 122 of the inner shaft 116. Divertors 134 may also be paced on an inner surface of the suction bore 132, such as and without limitation proximal to the inner hub 120. Instead of, or in addition to, divertors 134, rifling may be placed inside the channel 126, such as and without limitation in an area proximal to the distal end 122 of the inner shaft 116. Additionally, rifling ma also be placed on an inner surface of the suction bore 132, such as and without limitation proximal to the inner hub 120.

There is disclosed in the above description and the drawings, a surgical device system that fully and effectively overcomes the disadvantages associated with the prior art. However, it will be apparent that variations and modifications of the disclosed implementations may be made without departing from the principles described herein. The presentation of the implementations herein is offered by way of example only and not limitation.

Any element in a claim that does not explicitly state “means” for performing a specified function or “step” for performing a specified function, should not be interpreted as a “means” or “step” clause as specified in 35 U.S.C. § 112.

What is claimed is:

1. A surgical device comprising:
a body further comprising a proximal end and a distal end;
a hollow cannula extending from the distal end of the body, the cannula further comprising a proximal end and a distal end;
an operative head positioned at the distal end of the cannula; and
a suction connector in fluid communication with the hollow cannula, the suction connector being configured for connection to a suction source;
wherein the hollow cannula further comprises at least one of the group consisting of diverters and rifling on at least a portion of an inside surface.

2. The surgical device of claim 1 wherein the operative head further comprises a radio frequency ablator.

3. The surgical device of claim 1 wherein the operative head further comprises diverters on at least a portion of an inside surface.

4. The surgical device of claim 1 wherein the operative head further comprises rifling on at least a portion of an inside surface.

5. The surgical device of claim 1 wherein the body further comprises a suction bore extending from the suction connector to a position near the hollow cannula, the suction bore being in fluid communication with the suction connector and the hollow cannula; and wherein the suction bore further comprises diverters along at least a portion of an inside surface.

6. The surgical device of claim 1 wherein the body further comprises a suction bore extending from the suction connector to a position near the hollow cannula, the suction bore being in fluid communication with the suction connector and the hollow cannula; and wherein the suction bore further comprises rifling along at least a portion of an inside surface.

7. The surgical device of claim 1 wherein the hollow cannula comprises diverters on at least a portion of an inside surface and wherein the diverters further comprise a round profile.

8. The surgical device of claim 1 wherein the hollow cannula comprises diverters on at least a portion of an inside surface and the diverters are in a helical pattern.

9. The surgical device of claim 1 wherein the hollow cannula comprises diverters on at least a portion of an inside surface and the diverters are in a double helix pattern.

10. The surgical device of claim 1 wherein the hollow cannula comprises rifling on at least a portion of an inside surface; and wherein the rifling is in a helical pattern.

11. The surgical device of claim 1 wherein the hollow cannula comprises rifling on at least a portion of an inside surface; and wherein the rifling is in a double helix pattern.

12. A surgical device comprising:
a body further comprising:
a proximal end and a distal end;
a suction connector coupled to the body, the suction connector being configured for connection to a suction source;
a hollow cannula extending from the distal end of the body, the cannula further comprising a proximal end and a distal end;
an inner shaft rotatably positioned within the hollow cannula, the inner shaft further comprising a proximal end, a distal end, and a channel;
an inner drive hub coupled to the proximal end of the inner shaft, the inner drive hub being rotatably coupled to the body and in fluid communication with the suction connector;
wherein the channel further comprises at least one of the group consisting of diverters and rifling on at least a portion of an inside surface.

13. The surgical device of claim 12 wherein a suction bore extends from the suction connector to a position near the inner hub, the suction bore being in fluid communication with the suction connector and the channel of the inner shaft; and wherein the suction bore further comprises diverters along at least a portion of an inside surface.

14. The surgical device of claim 12 wherein a suction bore extends from the suction connector to a position near the inner hub, the suction bore being in fluid communication with the suction connector and the channel of the inner shaft; and wherein the suction bore further comprises rifling along at least a portion of an inside surface.

15. The surgical device of claim 12 wherein the channel comprises diverters on at least a portion of an inside surface and wherein the diverters further comprise a round profile.

16. The surgical device of claim 12 wherein the channel comprises diverters on at least a portion of an inside surface and wherein the diverters are in a helical pattern.

17. The surgical device of claim 12 wherein the channel comprises diverters on at least a portion of an inside surface and wherein the diverters are in a double helix pattern.

18. The surgical device of claim 12 wherein the channel comprises rifling on at least a portion of an inside surface and wherein the rifling is in a helical pattern.

19. The surgical device of claim 12 wherein the channel comprises rifling on at least a portion of an inside surface and where the rifling is in a double helix pattern.

20. The surgical device of claim 12 further comprising:
a cutting head positioned at the distal end of the hollow cannula; and
a blade positioned at the distal end of the inner shaft;
wherein as the inner shaft is rotated relative to the cannula the blade cooperates with the cutting head to cut tissue in contact with the cutting head.

21. The surgical device of claim 12 wherein the surgical device is configured as a shaver, drill, burr or rasp.
